(19) United States
(12) Patent Application Publication
(10) Pub. No.: US 2014/0161915 A1
(43) Pub. Date: Jun. 12, 2014

(54) Solubilization of cucurminoid compounds and products thereof

(71) Applicants: Adam J. Payne, Edmond, OK (US); Biji T. Kurien, Yukon, OK (US); R. Hal Scofield, Oklahoma City, OK (US)

(72) Inventors: Adam J. Payne, Edmond, OK (US); Biji T. Kurien, Yukon, OK (US); R. Hal Scofield, Oklahoma City, OK (US)

(21) Appl. No.: 13/694,368

(22) Filed: Nov. 26, 2012

Related U.S. Application Data
(60) Provisional application No. 61/629,738, filed on Nov. 26, 2011.

(51) Int. Cl. A61K 36/9066; G01N 33/58; A23L 2/52; A23L 1/272; A23L 1/221; A61K 31/12; A23L 1/30

(52) U.S. Cl. CPC A61K 36/9066; A61K 31/12; G01N 33/583; A23L 1/3002; A23L 1/272; A23L 1/221; A23L 2/52
USPC 424/756; 514/679; 436/86

(57) ABSTRACT

The invention provides a method for dissolving curcumin in aqueous solution at enhanced concentrations and with enhanced stability against precipitation and degradation, and provides solutions thereof; the invention further provides methods of use for them.

### RELATED APPLICATIONS

This application claims priority to provisional U.S. patent application No. 61/629,738, having the same title and filed Nov. 26, 2011.

### FIELD OF THE INVENTION

This invention pertains to aqueous solutions of curcuminoids and their use for nutraceutical and pharmaceutical formulations.

### BACKGROUND

Turmeric is a curry spice obtained from the rhizome of Curcuma longa of the ginger family, and has a long history of use in ayurvedic medicine and in traditional Asian diets. The principal bioactive component of turmeric is curcumin (1,7-bis[4-hydroxy-3-methoxyphenyl]-1,6-heptadiene-3,5-dione), a yellow pigment first identified in 1910. Curcumin is used widely in the United States for food coloring (e.g, in mustard, cheese, spices, cereals, potato flakes, soups, pickles, ice cream, and yogurt), but also as a nutraceutical. The interest in turmeric and its molecular components is rising. Curcumin in particular has become an important focus of pharmaceutical development. A June 2011 report stated that 61 clinical trials registered with the U.S. National Institutes of Health were completed or underway on the use of dietary curcumin in treating a variety of clinical disorders.

Structurally curcumin exists as a tautomer; the enol form tends to predominate both in solution and in the solid phase.

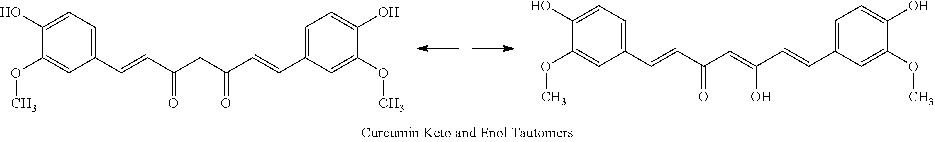 [SMILES: COc1cc(/C=C/C(=O)CC(=O)/C=C/c2ccc(O)c(OC)c2)ccc1O ; COc1cc(/C=C/C(=O)/C=C(O)/C=C/c2ccc(O)c(OC)c2)ccc1O]

Commercial curcumin contains curcumin itself (77%), desmethoxycurcumin (DMC, 17%) and bisdesmethoxycurcumin (BDMC, 3%); the latter two differ from curcumin only by lacking one or both methoxy groups, respectively. As a group these compounds and their derivatives for pharmaceutical use are referred to as curcuminoids. Among the major cellular metabolites of these three compounds are the tetrahydrocurcuminoids, in which both vinylidene groups are reduced, i.e., THC (i.e., tetrahydrocurcumin), TDMC and TBDMC. The tetrahydrocurcuminoids retain the bioactivity but are colorless and more chemically stable than the curcuminoids.

Curcumin's medicinal properties include substantial effects that are antioxidative, anti-inflammatory, antiviral, antibacterial and antifungal, and it is safe for human consumption even at the level of 8 g/day ingestion for sustained periods. Studies have shown that to varying degrees curcumin promotes wound healing and has therapeutic and or preventive effects against diabetes, asthma, allergies, cataracts, atherosclerosis, Alzheimer's disease, Parkinson's disease, myelodysplastic syndromes, cystic fibrosis, myocardial infarctions, high cholesterol, stroke, malaria, HIV, HSV-1, psoriasis, and others. Among the diseases for which curcumin has ameliorative effects are autoimmune diseases, including multiple sclerosis, rheumatoid arthritis, psoriasis, inflammatory bowel disease, Sjögren's syndrome, systemic lupus erythematosus, type I diabetes mellitus, neurodegenerative diseases, and several types of cancer.

Curcumin's efficacy is thought to arise in part from its blocking the effects of 4-hydroxy-2-nonenal (HNE), an alpha, beta-unsaturated aldehyde that is an oxidation byproduct from disease pathogenesis and that covalently modifies cellular targets; HNE is cytotoxic, mutagenic and genotoxic. Curcumin also suppresses the expression of c-jun, a proliferation-stimulating gene in immune cells; this affects both apoptosis and cancer. In addition curcumin has also been shown to bind in both the minor and major grooves of DNA and RNA as well as at their phosphate groups, yet without being a mutagen. Curcumin binds to proteins nonspecifically and inhibits their oxidative modification. Curcumin's benefits against autoimmune diseases appear to be linked to its ability to regulate inflammatory cytokines such as IL-1beta, IL-6, IL-12, TNA-alpha, and IFN-a, and associated JAK-STAT, AP-1 and NF-kB signaling pathways in immune cells, and may also arise from inhibiting unwanted antibody-antigen interactions.

Available data suggests that individual curcuminoids differ in their effects for important biological activities and in some cases are synergistic with each other. For instance, when individual curcuminoids are used at high purity (e.g., 95-99%) differences exist between their respective anti-proliferative effects against cancer cell lines, and in fact a mixture of the curcuminoids is more effective at suppressing nuclear factor-kB (NF-kB) activation in the inflammation response than are the components in isolation. Curcuminoids also induce glutathione S-transferase and are potent inhibitors of cytochrome P450.

Nevertheless the use of purified curcumin is often preferred because it is a potent antioxidant (including against superoxide and singlet oxygen) for multiple targets, is non-toxic up to 12 g/day, and can regress a variety of disorders inexpensively. Curcumin scavenges free radicals and inhibits lipid peroxidation and oxidative DNA damage. Curcumin is a chemopreventive agent for cancers of the colon, prostate, esophagus, lung, breast and mouth. It has been used in inhibiting viral and bacterial growth as well as for atherosclerosis. Curcumin suppresses cyclin D1 and anti-apoptotic gene products to yield anti-proliferative effects. Very recently curcumin was shown to suppress NF-kB and increase apoptosis in both Flo-1 and OE33 esophageal adenocarcinoma (EAC) cell lines. and was shown to enhance both 5-fluorouracil and cisplatin-mediated chemosensitivity in EAC. Curcumin conjugated with polyethylene glycol (35 kD) displayed greater reduction of cell growth compared to free curcumin in pancreatic cancer cells, by affecting cell cycle progression owing to its better solubility in water. The results of this study suggest that PEGylated curcumin inhibits cell proliferation through suppression of Jun activation domain binding protein 1/COP9 signalosome complex activity. Curcumin also induces cytochrome C release, caspase activation, p53 activation, and is anti-angiogenic by down-regulation of vascular endothelial growth factor (VEGF).

Medicinal use of curcumin has been limited because the compound is virtually insoluble (dissolution of only ca. 600 ng/mL) in water at acidic and physiological pH and hydrolyzes rapidly at alkaline pH. Plasma concentrations from high human oral doses (8-12 g/day) of curcumin are just in the nanomolar range. Most therapeutic in vitro studies on curcumin have solubilized it in organic solvents. Those solvents include dimethylsulfoxide (DMSO), acetone and ethanol but they have limited suitability for in vivo studies themselves. Surfactants or co-surfactants can be used to improve solubility in water, but are often undesirable for other reasons. The curcuminoids also have poor solubility in lipids. Curcumin has been solubilized by sulfation and glucuronidation, however those derivatives cannot penetrate the blood brain barrier whereas the parent compound can. And other derivatives cause concerns because their effects and side effects are not as well known.

Sabinsa Corp. has used piperine (“bioperine” extracted from black pepper) to increase the solubility of curcumin, but this effect lasts only one hour. (Shoba G; Joy D; Joseph T; Majeed M; Rajendran R; Srinivas P S (May 1998). “Influence of piperine on the pharmacokinetics of curcumin in animals and human volunteers”. Planta Med 64 (4): 353-6).) Formulations in lipids or phospholipids improve bioavailability of the free curcumin, and are used in products such as Longvida Optimized Curcumin (achieving 5 μM in the brain), Meriva® (little or no free curcumin in humans^{[60]}), and BCM-95® (includes turmeric oil, with mixed results). The use of cyclodextrin host-guest complexes, curcumin nanoparticles, curcumin complexes with albumin protein, and curcumin encapsulated in liposomes micelles (e.g, in poly(ethylene oxide)-poly(caprolactone) copolymers) have also been reported but not yet fully validated.

Some observers have concluded that solubility is not the only barrier for water as a delivery medium, reporting water-mediated hydrolysis of curcumin to vanillin and ferulic acid (Wang, Y J; Pan M H, Cheng A L, Lin L I, Ho Y S, Hsieh C Y, Lin J K. (1997). “Stability of curcumin in buffer solutions and characterization of its degradation products.”. J Pharm Biomed Anal. 15: 1867-76). Yet despite the degradation and relative insolubility of curcumin in aqueous solutions, the compound is most bioavailable at one of the body's major water ingesting sites, the colon. Indeed curcumin has some of its strongest observed medicinal effects there. Thus even trace levels of curcumin in water appear to have provided important medicinal value.

Consequently, there is an ongoing need for methods and processes that enhance curcumin's solubility in water in a stable way and provide pharmaceutical preparations at elevated concentrations of curcumin in water.

### BRIEF SUMMARY OF THE INVENTION

The present inventors have found that surprisingly both the aqueous concentration of curcumin and its stability against hydrolysis and precipitating out are enhanced to a very large degree by dissolving the curcumin at modestly elevated temperatures and pressures, that the effect is further enhanced by the inclusion of a small amount of an alcohol (only part of which is subsequently lost as vapor), and that the spectroscopic signature of the dissolved compound remains substantially identical. These results are counterintuitive and their molecular basis is not yet clear.

The inventors have also found that ingestion of aqueous solutions of curcumin that is solubilized in this manner provides substantial mood-elevating benefits for a period of several hours.

In one embodiment, the invention provides a process for solubilizing and stabilizing curcumin in an aqueous solution, comprising:
a) providing a quantity of water;
b) providing over 0.008 mg curcumin per mL water;
c) optionally providing at least 0.01 mL lower alcohol per mL of water;
d) optionally providing at least 0.01 mL polyalkyleneoxide per mL of water;
e) combining the water, curcumin and optional lower alcohol in any order and optionally with mixing; and
f) heating the combined water, curcumin and optional lower alcohol to a temperature in excess of 100%, optionally at a pressure exceeding 1 atmosphere;
   wherein steps a), b) and c) may be done in any order, and wherein the process includes at least one of combining the lower alcohol or optional polyalkyleneoxide in step e) and applying a pressure exceeding 1 atmosphere in step f).

In a further embodiment, the invention provides a product comprising solubilized and stabilized curcumin in an aqueous solution made by a process comprising solubilized and stabilized curcumin in an aqueous solution made by a process comprising:
a) providing a quantity of water;
b) providing over 0.008 mg curcumin per mL water;
c) optionally providing at least 0.01 mL lower alcohol per mL of water;
d) optionally providing at least 0.01 mL polyalkyleneoxide per mL of water;
e) combining the water, curcumin, optional lower alcohol and optional polyalkyleneoxide in any order and optionally with mixing; and
f) heating the combined water, curcumin and optional lower alcohol to a temperature in excess of 100%, optionally at a pressure exceeding 1 atmosphere;
   wherein steps a), b) and c) may be done in any order, and wherein the process includes at least one of combining the lower alcohol or optional polyalkyleneoxide in step e) and applying a pressure exceeding 1 atmosphere in step f).

In a particular embodiment the invention provides a method of treatment comprising administering to a subject in need thereof an aqueous solution comprising over 0.008 mg curcumin per mL water to treat, prevent or ameliorate a medical or psychological condition.

In an additional embodiment the invention provides a process for staining proteins comprising:
a) providing a solution of curcumin that has been solubilized by heating in water, optionally under pressure and optionally in the presence of a lower alcohol or a polyalkyleneoxide;
b) combining the solubilized curcumin with one or more proteins in an assay for an amount of time sufficient to stain the protein; and
c) determining a presence or amount of protein as a function of the fluorescence or colorimetric density of the stain.

### DESCRIPTION OF THE DRAWINGS

FIG. 1 provides a histogram depicting the effects of heat, pressure and ethanol in solubilization of curcumin in water according to the invention.

### DETAILED DESCRIPTION OF THE INVENTION

### Previous State of the Art for Aqueous Solutions:

Prior mixtures that had 5 mg of solid curcuminoids added per mL of deionized water at room temperature typically resulted in dissolution of only about 0.13% of the curcuminoid mass. Given curcumin's molecular mass of 368.38, that represents 0.017 mM solutions. Yet essentially all of the dissolved material is DMC and BDMC; curcumin itself remains insoluble under these conditions (which suggests that prior studies studying the effects of aqueous “curcumin” did not in fact evaluate the effects of curcumin at all). For solutions made at room temperature it is difficult to obtain a clear (i.e., solid-free) supernatant even from careful centrifuging. The samples are far more soluble in alkaline water (e.g., pH 7.6), but when placed in phosphate buffered saline (PBS) and serum-free media at modest alkaline pH (pH 7.2, at 37° C.) the curcuminoids are hydrolyzed substantially completely within 30 minutes.

Two of the present inventors reported in 2007 that heat treatment increased curcumin's solubility in pure water. (B. B. Kurien, A. Singh, H. Matsumoto and R. H. Scofield, “Improving the Solubility and Pharmacological Efficacy of Curcumin by Heat Treatment,” ASSAY and Drug Dev. Technol., 5(4):567-576 (2007). There the curcumin was held in boiling deionized water. The maximum heat-dissolved amount increased from about 0.13% of the total amount of curcumin present (for unheated solutions) to 1.5% (ca. 75 μg/mL, 0.20 mM for heated solutions). This represented a 12-fold increase in solubility; the solubility increase for turmeric generally was 3-fold. After the solution was centrifuged to remove the undissolved material, none of the dissolved curcumin was observed to precipitate out over time. Moreover, instead of hydrolyzing rapidly as for curcumin solutions prepared at room temperature, the boiled curcumin solutions had only 47% hydrolysis after 12 hours and only 67% hydrolysis after 72 hours of exposure to alkaline pH (pH 7.2, at 37° C., in PBS). Even so, UV-Visible spectra and mass specta (from matrix assisted laser desorption deionization time of flight mass spectrometry, MALDI-TOF-MS) confirmed that the dissolved curcumin was substantially unchanged by the heat treatment. Heat treatment also appears to increase the solubilization of BDMC but (curiously) not DMC.

The curcumin samples in that report were obtained from Sigma Chemical Co. and Cayman Chemical. Following heating of the samples in deionized water they were centrifuged at 1000 g to remove insoluble particles, then centrifuged twice at 26,895 g (30 minutes each time) to remove residual insoluble curcumin particles. As necessary aliquots from these samples were re-centrifuged at 16,200 g for 20 minutes until the supernatant was clear; this liquid was used to determine curcumin concentrations using a UV-Vis spectrometer at 405 nm, with a water blank for comparison.

The heat-solubilized curcumin was validated by confirming that it inhibited 4-hydroxy-2-nonenal-mediated oxidative modification of a protein substrate by 80%. We also confirmed that autoantibodies human subjects treated with heat-solubilized curcumin/turmeric significantly inhibited binding of anti-Ro60 autoantibodies from systemic lupus erythematosus (SLE) (up to 52/70%, respectively) and Sjögren's syndrome sera to Ro60 autoantigen (up to 43/70%, respectively) in a non-specific way. In addition, we confirmed that the heat-solubilized curcumin/turmeric also inhibited binding of commercial polyclonal anti-spectrin to spectrin (50/56%, respectively) and binds to proteins on SDS-PAGE media, thus it is not specific to autoimmune diseases. Interestingly we found that heat solubilized curcumin does not intercalate into or otherwise bind to DNA, and in part that validates its safety for medicinal use.

### Solubilization Advances of the Present Invention:

That prior-reported heat dissolution method appeared to have reached the natural limit for dissolving curcumin in water because no benefit resulted from providing more than 5 mg of curcumin powder per mL water to be heated, nor in boiling the curcumin for more than 10 minutes. However the inventors have now discovered that using an autoclave to heat aqueous mixtures having curcumin present at 5 mg/mL substantially increases the amount of curcumin that can be dissolved. Thus treating the samples at 15 psi (i.e., ca. 1.02 atm) pressure and 121° C. and then centrifuging the autoclaved solutions (to remove the undissolved mass) yields samples with dissolution increased to 5% of the curcumin initially present (ca. 250 μg/mL, 0.68 mM). The autoclave cycle was 70 minutes, of which ˜40 minutes represent actual time under pressure. The pressure-dissolved curcumin was confirmed by UV-Vis spectroscopy to be unmodified, and did not precipitate out or cause the solution to become cloudy or turbid over time, thus it appears these are stable solutions. Exposure to alkaline pH for 72 hours led to only a 32% reduction in pristine curcumin. The increase in stability over solutions prepared at ambient temperature suggests that the heat- or pressure-dissolved curcumin might be present in a previously unrecognized ordering of molecules, however the invention is not so limited.

The inventors have further discovered that the curcumin concentration can be improved still more and disproportionately by initially including up to 5% of an alcohol such as methanol, ethanol, iso- or n-propanol, n-, sec- or tert-butanol or another alcohol when making up the initial mixture for pressurization. In an illustrative example, 2.5 mL of neat ethanol was combined with 250 mg of curcumin, resulting in a turbid mixture. That mixture was then combined with 47.5 mL of deionized water to make up a 50 mL solution. Some liquid is lost during autoclaving, however more is lost when methanol or ethanol is included than when water is the only liquid medium; it appears that some but not all of the alcohol is lost as vapor from the mixture; due to azeotrope effects and it is believed that a trace amount or more of alcohol may remain. The alcohol content can be further reduced by further exposure to vacuum pressures if desired. For exactness in quantification, if the solution is made up to the full 50 mL again by replacing lost fluid with only water, and if the undissolved curcumin is removed by centrifuging, the resulting solution has about 60 μg/mL curcumin dissolved, or about 12.9% of the original amount, which represents about a 100-fold increase over curcumin dissolved at ambient temperature, to about a 1.7 mM solution. Using larger amounts of ethanol (e.g., doubling its proportion) provided little improvement. UV-Vis spectra again confirmed the undegraded nature of the curcumin.

Note that when evaporation losses of the autoclaved solution are not replaced the concentrations of the obtained solutions are higher by several percent than the numbers just reported.

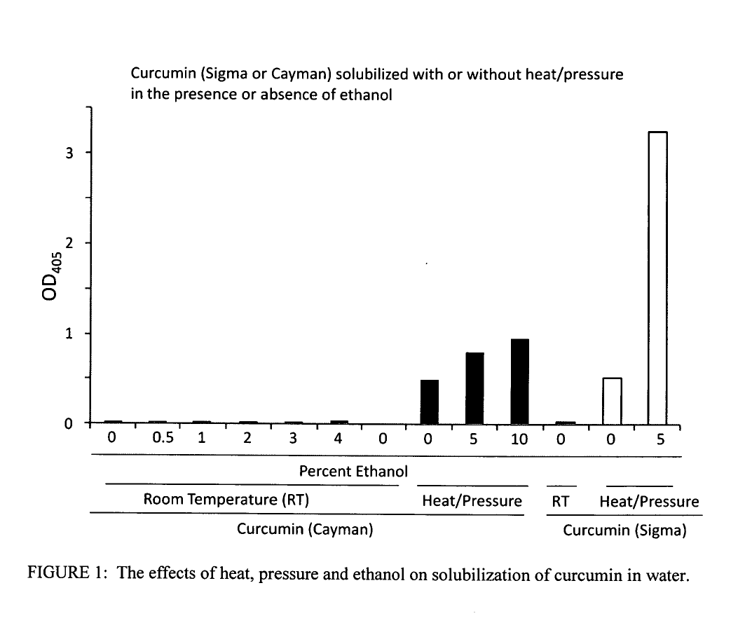

FIG. 1 provides some data in a visual format from these preparations. The OD₄₅₀ values on the y-axis pertain to the concentration of dissolved curcumin observed as a function of the absorption of light at 405 nm. The numerical values on the x-axis pertain to the percentages of ethanol in the aqueous medium (prior to any heating). The bar heights of the histogram represent the relative concentrations observed. “Heat/Pressure” refers to use of the autoclave protocol for solubilization. RT represents room temperature. Cayman and Sigma pertain respectively to fine chemical suppliers of curcumin, from Cayman Chemical (solid bars) and Sigma Chemical Co. (unfilled bars); the differences in the result are presumably due to differences in the composition of their natural extracts.

It has further been discovered that the inclusion of a trace amount of a poly(alkylene ether) solubilizes curcumin and its derivatives still further as shown below in the table. Autoclaving curcumin in water with 2% of a poly(alkyl ether) solubilizes 31% of 5 g of starting curcumin in a 50 mL starting solution. Only 920 μL of a 0.5% Tween-20 autoclaved solution is needed to attain a target in vivo level of 51 ng/mL in an average subject's plasma (i.e., 5 L), and only 160 μL of an autoclaved 2% Tween-20 solution of curcumin are needed per subject plasma liter.

| Curcumin Dissolution Protocol | Dissolution (Approximate) | |
|---|---|---|
| (Saturated) | Mass/mL | Molarity |

Prior Art Methods

| | | | | |
|---|---|---|---|---|
| Prior art: deionized water | 6.5 | μg/mL | 0.017 | mM |
| at room temperature | | | | |

| | | | |
|---|---|---|---|
| Kurien et al. (2007): boiling | 75 μg/mL for | 0.20 | mM |
| deionized water | heated solutions | | |

Illustrative Examples from Invention

| | | | | |
|---|---|---|---|---|
| 15 psi, 121° C., deionized | 250 | μg/mL | 0.68 | mM |
| water | | | | |
| 15 psi, 121° C., 5% ethanol | 640 | μg/mL | 1.7 | mM |
| in deionized water | | | | |
| 0.1% Tween-20, room pressure, | 22.5 | μg/mL | 0.061 | mM |
| 100° C. deionized water | | | | |
| 0.1% Tween-20, 15 psi, | 44.84 | μg/mL | 0.12 | mM |
| 121° C., deionized water | | | | |
| 0.5% Tween-20, room pressure, | 77.44 | μg/mL | 0.21 | mM |
| 100° C. deionized water | | | | |
| 0.5% Tween-20, 15 psi, | 278.1 | μg/mL | 0.75 | mM |
| 121° C., deionized water | | | | |
| 2% Tween-20, room pressure, | 443.52 | μg/mL | 1.2 | mM |
| 100° C. deionized water | | | | |
| 2% Tween-20, 15 psi, | 1556 | μg/mL | 4.2 | mM |
| 121° C., deionized water | | | | |

The availability and bioactivity of curcumin dissolved according to the invention were found to have surprising efficiency in staining proteins. The proteins were presented in gel lanes having 2 μg, 1 μg, 500 ng, and 100 ng of protein marker and compared to the staining rate of Coomassie Brilliant Blue (CBB). Staining solutions were used directly from the samples prepared as above by autoclaving or by heating to 100° C., except that solubilized curcumin solutions that were prepared with Tween additives were diluted 1:50 with water due to their high concentration of curcumin.

The curcumin that was solubilized according to the invention in either pristine form or from turmeric stained the proteins with almost the same sensitivity as CBB did. Unlike the four hours required for staining with CBB, the curcumin and turmeric stains required only 30 minutes, though staining tests were done for as long as two hours. Moreover the staining of the invention is suitable for calibration by ordinary means for fluorescent and colorimetric dyes. The results of the invention are quite surprising because curmurin that had been solubilized by ethanol, DMSO or alkali was essentially ineffective for staining proteins. For the curcumin that had been solubilized in DMSO the DMSO solution was dissolved with water to obtain a 0.1% DMSO solution. The proteins employed included IgM, Fc fragment, interleukin 13 receptor, mouse serum and bovine serum albumin; all were stained by solutions of the invention.

Curcumin and turmeric that are solubilized according to the invention are unique and ideal in that not only does their staining efficiency approach that of CBB, but they require no de-staining, and they are nontoxic and environmentally friendly. The new stains can be discarded in the sink, unlike CBB. Moreover the price of turmeric is less than 1% that of CBB.

References are cited at the end of this description, the teachings of all of which are incorporated herein by reference.

### Pharmacodynamic Advances of the Present Invention:

These nanomolar solutions were found to have surprising pharmacodynamic efficacy and entirely unexpected effects. The phenomena were observed consistently in four adult males (human), who each reported unexpected mood elevating effects that manifested almost instantaneously and lasted four or more hours. The study conditions were as follows.

Subject #1 had a body mass of about 60 kg, and ingested 5 mL of a 350 micromolar sample of curcumin that had been prepared three days earlier using the autoclave with 5% ethanol as described above and adjusted to the final concentration.

Subject #2 had a body mass of about 60 kg, and ingested 10 mL of a 350 micromolar sample of curcumin that had been prepared three days earlier using the autoclave with 5% ethanol as described above and adjusted to the final concentration.

Subject #3 had a body mass of about 100 kg, and ingested 20 mL of a 350 micromolar sample of curcumin that had been prepared three days earlier using the autoclave with 5% ethanol as described above and adjusted to the final concentration. In addition to the mood elevating results this subject experienced reproducible relief from metatarsal arthritic inflammation for a period of about 48 hours; anti-inflammatory effects were already a known property of curcumin.

Subject #4 had a body mass of about 60 kg, and ingested about 8.5 mL of a 150 micromolar sample of curcumin that had been prepared five days earlier as described above using the autoclave but not the, and adjusted to the final concentration. This subject's study was conducted in a completely blind manner: the subject was told nothing about the known or suspected properties but was merely asked to report his observations.

### DEFINITIONS

The invention may be better understood by reference to the following definitions for the terms as used herein.

The term “solubilizing” means making a solute or potential solute soluble or more soluble in a liquid.

The term “solute” means a substance that is dissolved in a liquid; the term “potential solute” means a substance that is to be dissolved in a liquid.

The term “stabilizing” means altering conditions such that a particular solute is more stable against degradation in a given solution.

The term “aqueous” means a liquid comprising a substantial proportion of water.

The term “solution” means a liquid comprising a second liquid and or comprising one or more solutes.

The term “lower alcohol” is used in the sense of organic chemistry and means an alcohol having from one to four covalently bonded carbons. The term “alcohol” as used herein includes saturated and unsaturated alcohols, linear and cyclic alcohols, and branched carbon chain alcohols. The term “alcohol” as used herein includes alcohols substituted by one or more halogens. The term “lower alcohol” as used herein optionally includes mixture of lower alcohols. Illustrative non-limiting examples of linear, cyclic and branched saturated alcohols without halogen substitution include methanol, ethanol, n-propanol, isopropanol, cyclopropanol, n-butanol, sec-butanol, tert-butanol, cyclobutanol, n-hydroxymethyl(cyclopropane), sec-hydroxy(methylcyclopropane) and tert-hydroxy(methylcyclopropane). As used herein with respect to alcohols, the term “low boiling” means boiling at a temperature below 100° C.

The terms “poly(alkylene ether),” “polyalkyleneoxide” and “polyoxyalkylene” as used herein are synonymous and used in the sense of organic and polymer chemistry; they refer to a polyether such as an oligomer, polymers of any molecular weight of polyoxyethylene, polyoxypropylene, polyoxybutylene, polyoxypentylene, polyoxyhexylene, pegylated species such as the Tween series of polysorbates, and other poly(alkylene ether)s. The terms are not limited by the type of end groups on the respective structure; the end groups may be hydroxyl, alkyl ether, carboxylic acid, carboxylic ester, or other end groups such as are known in the art for polyoxyalkylenes.

The term “food product” means a substance that is a food or beverage, or that is a condiment, glaze, flavoring, coloring, supplement, or other ingredient or additive for a food or beverage.

The term “supplement product” means a non-pharmaceutical product intended for ingestion to facilitate the health of a subject by natural means, optionally at the subject's discretion.

The term “pharmaceutical product” means a product intended for use by medical prescription or for use as an over-the-counter drug.

### Example 1

### Water: Curcumin Ratios

A solution of deionized, distilled, or other quality of water is provided in an amount of 40-50 mL. The invention is not limited by the amount of water provided. A sample comprising curcumin is also provided. In preferred embodiment the sample comprises a sufficient amount of curcumin that if all of it was dissolved the curcumin concentration would exceed 0.008 mg/mL. In particular embodiments, total dissolution of the sample in the water would result in a curcumin concentration exceeding any of 0.015 mg/mL, 0.023 mg/mL, 0.030 mg/mL, 0.038 mg/mL, 0.045 mg/mL 0.052 mg/mL, 0.060 mg/mL, 0.090 mg/mL, 0.12 mg/mL, 0.15 mg/mL, 0.18 mg/mL, 0.24 mg/mL, 0.30 mg/mL, 0.40 mg/mL. 0.50 mg/mL, 0.60 mg/mL, 1.20 mg/mL, 1.80 mg/mL, 2.40 mg/mL and 3.00 mg/mL.

### Example 2

### Alcohol

A lower alcohol is provided, wherein lower means having four carbons or less, which may be: saturated or unsaturated; linear, branched or cyclic; and substituted or not by a halogen. The alcohol optionally may be a mixture of lower alcohols. In particular embodiments the alcohol is low boiling but the invention is not so limited.

### Example 3

### Poly(alkylene ether)

A polyalkyleneoxide (PAO) is provided, wherein the ether oxygen atoms are separated by an from two to six carbons in the backbone of an alkylene group, and the alkylene group may be: saturated or unsaturated; linear, branched or cyclic; and substituted or not by a halogen. The PAO optionally may comprise end groups that are hydroxyl, ether, alkyl, or carboxylic ester, and may comprise internal groups that are ether, alkyl or carboxylic ester. The PAO optionally may comprise a mixture of PAO compounds, and optionally may be a single PAO compound comprising a mixture of alkylene groups in its structure. In particular embodiments one or more of the PAO ether oxygens may be part of an etherified sugar moiety such as in a polysorbate compound.

### Example 4

### Unheated Mixtures

The water and sample comprising curcumin from Example 1 and optionally the lower alcohol from Example 2 or PAO from Example 3 are combined in any order desired, optionally with mixing.

If the alcohol is included, it is provided in a proportion up to 75% of the total liquid, optionally 1-50% of the total liquid, optionally 2-25% of the total liquid, optionally 3-15% of the total liquid, optionally 4-10% of the total liquid, or about 5% of the total liquid.

If the PAO is included, it is provided in a proportion up to 10% of the total liquid, optionally 0.1-8.0% of the total liquid, optionally 0.2-6.0% of the total liquid, optionally 0.5-4.0% of the total liquid, optionally 1-3% of the total liquid, or about 2% of the total liquid.

### Example 5

### Heated Mixtures

The combination from Example 4 is heated. The maximum temperature may be in the range 105-300° C., 110-250° C., 115-200° C., 120-150° C., or about 125° C.

The heating is optionally carried out under pressure. The maximum pressure may be in the range 1-220 atm, 1-100 atm, 1.01-50 atm, 1.01-10 atm, 1.02-5 atm, 1.02-2 atm, or between 1 and 1.2 atm.

The duration of heating is optionally at least 10 seconds, 1 minute to 72 hours, 5 minutes to 24 hours, 10 minutes to 12 hours, 15 minutes to 6 hours, 20 minutes to 3 hours, 30 minutes to 1 hour, or about 30 minutes.

In a particular embodiment the maximum temperature is 121° C., at a pressure of 15 psi (1.02 atm) for between 30 and 60 minutes.

In particular embodiments the heating is carried out in an autoclave, a bomb calorimeter, or a sealed reaction vessel. In some embodiments the heating is conducted in a reaction vessel under high pressure from an applied gas such as: steam; a noble gas; a relatively inert gas such as carbon dioxide, carbon monoxide, dinitrogen, or another relatively inert gas; a reducing gas such as dihydrogen or another reducing gas; or another gas under pressure.

In certain embodiments the heating is carried out in an unstoppered tube, flask, beaker or other open reaction vessel that is capable of allowing a liquid within it to evaporate and exit the reaction vessel during the heating.

In still further embodiments mixtures that have been heated are allowed to cool before further processing.

### Example 6

### Volume Gross Up

In certain embodiments the mixtures from Example 5 after heating optionally receive additional amounts water or a lower alcohol to replace fluids lost during heating. In certain embodiments the replacement brings solutions approximately exactly up to their original volumes. In other embodiments the replacement brings total solutions to less than their original volumes. In further embodiments the replacement brings total solutions to more than their original volumes. In still other embodiments liquid is removed from the mixtures after their heating by pouring, static evaporation, lyophilizing, use of molecular sieves, or other means.

### Example 7

### Separation

In certain embodiments the mixtures from Example 5 or Example 6 after heating are used for medicinal or food purposes without separation of any remaining undissolved material.

In other embodiments the mixtures from Example 5 after filtered by means of a filtration apparatus such as a paper filter, ceramic filter, glass frit or other filtration apparatus known to persons of ordinary skill in the art to remove undissolved solids, and optionally this is done with the assistance of a pump to push the liquid through the filtration apparatus or a vacuum line to pull the liquid through the filtration apparatus.

In particular embodiments the mixtures from Example 5 are filtered by means of dialysis membranes to remove undissolved solids.

In other embodiments residual solids in the mixtures from example 5 are separated by means of centrifugation and pouring off the supernatant.

### Example 8

### Ingested Supplements

In certain embodiments a filtered or unfiltered mixture described in Example 7 is incorporated in a food product. The food product is optionally a beverage, condiment, coloring, glaze, spice, herbal mixture, or other food product. In particular embodiments a mixture cited in Example 7 is incorporated into a liquid nutraceutical supplement, or into a nutraceutical supplement that has a liquid component such as a gel or capsule.

### Example 9

### Indications

In certain embodiments a filtered or unfiltered mixture cited in Example 7 is provided to a subject in need thereof to prevent, treat, or ameliorate a medical condition. In particular embodiments the condition is selected from one that requires an antioxidative, anti-inflammatory, antiviral, antibacterial and or antifungal effect. In certain embodiments the condition is an autoimmune disease such as multiple sclerosis, rheumatoid arthritis, psoriasis, inflammatory bowel disease, Sjögren's syndrome, systemic lupus erythematosus, type I diabetes mellitus, neurodegenerative diseases, or a type of cancer. In further embodiments the condition is one such as Type II diabetes, asthma, allergies, cataracts, atherosclerosis, Alzheimer's disease, Parkinson's disease, myelodysplastic syndromes, cystic fibrosis, myocardial infactions, high cholesterol, stroke, malaria, HIV, HSV-1, psoriasis, or another type of disease.

### Example 10

### Doses

In particular embodiments a mixture cited in Example 7 is provided to a consumer or subject in a quantity that provides up to 2, 4, 6, 8, 10, 12, 14 or 16 g/day of curcumin to a consumer of the product.

### Example 11

### Subjects

In some embodiments the subjects of Example 10 are human subjects. In certain other embodiments they are other mammalian subjects. In particular embodiments the subjects are selected from the group consisting of humans, pets, livestock, research animals and display animals, wherein these terms include but are not limited to: pets such as dogs, cats, rodents, birds, reptiles, amphibians, fish and other pets; livestock such as cattle, horses, sheep, goats, alpaca, chickens, geese, pigs, and other livestock; research animals such as mice, rats, pigeons, dogs, cats, captive woodland creatures, captive aquatic creatures, captive birds, captive desert creatures, captive marsh creatures and other research animals; and display animals such as zoo animals, carnival display animals, commercial aquarium animals, and other display animals.

### Example 12

### Psychodynamic effects

In some embodiments a mixture cited in Example 7 is provided to a subject of Example 11 for purposes of mood elevation. Illustrative but nonlimiting examples of subjects in need of mood elevation include clinically depressed subjects, bereaved subjects, subjects suffering physical pain, subjects exhibiting symptoms of dementia, and subjects under mental stress such as battlefield stress, academic stress, occupational stress, relational stress, and homesickness or other loneliness.

The foregoing description and drawings comprise illustrative embodiments of the preferred embodiment, but the invention is not so limited. It will be readily apparent to those of ordinary skill in the art that numerous alternatives, variations, modifications and permutations of the invention described herein may be made without departing from the spirit or nature of the invention; these are contemplated as being part of the invention. Also, merely listing or enumerating the steps of a method or the components of a device in a certain order does not constitute any limitation on the order of the steps or components of that method or device, respectively. Although specific terms are employed herein, they are used in a descriptive sense only and not for purposes of limitation.

### REFERENCES

1. Shishodia S, Chaturvedi M M, Aggarwal B B. Role of curcumin in cancer therapy. Curr Probl Cancer 31:243-305, 2007.
2. Lampe V, Milobedzka J. No title available. Ber Dtsch Chem Ces, 46:2235, 1913.
3. Sharma R A, McLelland H R, Hill K A., Ireson C R et al., Pharmacodynamic and pharmacokinetic study of oral Curcuma extract in patients with colorectal cancer. Clin. Cancer Res, 7:1894-1900, 2001.
4. Cheng A L, Hsu C H, Lin J K, Hsu M M. et al., Phase I clinical trial of curcumin, a chemopreventive agent, in patients with high-risk or pre-malignant lesions. Anticancer Res, 21:2895-2900, 2001.
5. Lao C D, Ruffin M T IV, Normolle D, et al. Dose escalation of a curcuminoid formulation. BMC Complement Ahern Med 6:10, 2006.
6. Dhillon N, Aggarwal B B, Newman R A, et al. Phase II trial of curcumin in patients with advanced pancreatic cancer. Clin Cancer Res, 14:4491-9, 2008.
7. Rowe D L, Ozbay T, O'Regan R M, Nahta R. Modulation of the BRCA1 protein and induction of apoptosis in triple negative breast cancer cell lines by the polyphenolic. Breast Cancer, 3:61-75, 2009.
8. Anto R J, George J, Babu K V et al Antimutagenic and anticarcinogenic activity of natural and synthetic curcuminoids. Mutat Res, 370:127-131, 1996.
9. Park B S, Kim J G, Kim M R, Lee S E, Takeoka G R, Oh K B, Kim J H. et al. Curcuma longa L. constituents inhibit sortase A and Staphylococcus aureus cell adhesion to fibronectin. J Agric Food Chem, 53:9005-9009, 2005.
10. Singh S, Aggarwal B B. Activation of transcription factor NF-kappa B is suppressed by curcumin (diferuloylmethane) [corrected]. J Biol Chem, 270:24995-25000, 1995.
11. Mukhopadhyay, A. et al. Curcumin-induced suppression of cell proliferation correlates with down-regulation of cyclin D1 expression and CDK4-mediated retinoblastoma protein phosphorylation. Oncogene, 21:8852-8861, 2002.
12. Hartojo W, Silvers A L, Thomas D G, Seder C W, Lin L, Rao H, Wang Z, Greenson J K, Giordano T J, Orringer M B, Rehemtulla A, Bhojani M S, Beer D G, Chang A C. Curcumin promotes apoptosis, increases chemosensitivity, and inhibits nuclear factor kappaB in esophageal adenocarcinoma, Transl Oncol 3:99-108, 2010.
13. Li J, Wang Y, Yang C, Wang P, Oelschlager D K, Zheng Y, Tian D A, Grizzle W E, Buchsbaum D J, Wan M. Polyethylene Glycosylated Curcumin Conjugate Inhibits Pancreatic Cancer Cell Growth through Inactivation of Jab 1. Mol Pharmacol, 76:81-90, 2009.
14. Gao, X. Gao X, Deeb D, Jiang H, Liu Y B, Dulchaysky S A, Gautam S C. Curcumin differentially sensitizes malignant glioma cells to TRAIL/Apo2L-mediated apoptosis through activation of procaspases and release of cytochrome c from mitochondria. J Exp Ther Oncol, 5:39-48, 2005.
15. Anto R J, Mukhopadhyay A, Denning K, Aggarwal B B. Curcumin (diferuloylmethane) induces apoptosis through activation of caspase-8, BID cleavage and cytochrome c release: its suppression by ectopic expression of Bcl-2 and Bcl-xl. Carcinogenesis, 23:143-150, 2002.
16. Liontas A, Yeger H. Curcumin and resveratrol induce apoptosis and nuclear translocation and activation of p53 in human neuroblastoma. Anticancer Res, 24:987-998, 2004.
17. Aggarwal B B, Sung B. Pharmacological basis for the role of curcumin in chronic diseases: an age-old spice with modern targets. Trends Pharmacol Sci, 30:85-94, 2009.
18. Bae M K, Kim S H, Jeong J W, Lee Y M, Kim H S, Kim S R, Yun I, Bae S K, Kim K W. Curcumin inhibits hypoxia-induced angiogenesis via down-regulation of HIF-1. Oncol Rep, 15:1557-1562, 2006.
19. Sandur S K, Pandey M K, Sung B, Ahn K S, Murakami A, Sethi G, Limtrakul P, Badmaev V, Aggarwal B B. Curcumin, demethoxycurcumin, bisdemethoxycurcumin, tetrahydrocurcumin and turmerones differentially regulate anti-inflammatory and anti-proliferative responses through a ROS-independent mechanism. Carcinogenesis, 28:1765-1773, 2007
20. Hoehle S I, Pfeiffer E, Solyom A M, Metzler M Metabolism of curcuminoids in tissue slices and subcellular fractions from rat liver. J Agric Food Chem, 54:756-764, 2006.
21. Jayaprakasha G K, Jena B S, Negi P S, Sakariah K K. Evaluation of antioxidant activities and antimutagenicity of turmeric oil: a byproduct from curcumin production. Z. Naturforsch. [C], 57:828-835, 2002.
22. Joshi J, Ghaisas S, Vaidya A, Vaidya R, Kamat D V, Bhagwat A N, Bhide S Early human safety study of turmeric oil (Curcuma longa oil) administered orally in healthy volunteers. J. Assoc. Physicians India, 51: 1055-1060, 2003.
23. Yodkeeree S, Chaiwangyen W, Garbisa S, Limtrakul P. Curcumin, demethoxycurcumin and bisdemethoxycurcumin differentially inhibit cancer cell invasion through the down-regulation of MMPs and uPA. J Nutr Biochem, 20:87-95, 2009.
24. An Na Kim, Woo-Kwang Jeon, Byung-Chul Kim, Up-regulation of heme oxygenase-1 expression through CaMKII-ERK1/2-Nrf2 signaling mediates the anti-inflammatory effect of bis-demethoxycurcumin in LPS-stimulated macrophages, Free Radical Biology and Medicine, 2010, doi:10.1016/j.freeradbiomed.2010.04.015
25. Kurien B T, Scofield R H. Autoantibody determination in the diagnosis of systemic lupus erythematosus. Scand. J Immunol, 64:227-235, 2006. Review.
26. Alarcon-Segovia D. The pathogenesis of immune dysregultion in systemic lupus erythematosus: a troika. J Rheumatol, 11:588-590, 1984.
27. Danchenko N, Satia J A, Anthony M S. Epidemiology of systemic lupus erythematosus: a comparison of worldwide disease burden. Lupus, 15:308-18, 2006. Review.
28. Scofield R H. Autoantibodies as predictors of disease. Lancet, 363:1544-46, 2004. Review.
29. Arbuckle M R, McClain M T, Rubertone M V, Scofield R H, Dennis G J, James J A, Harley J B. Development of autoantibodies before the clinical onset of systemic lupus erythematosus. N Engl J Med, 349:1526-33, 2003.
30. Harley J B, Sestak A S, Willis L G, Fu S M, Hansen J A, Reichlin M. A model for disease heterogeneity in systemic lupus erythematosus: Relationships between histocompatibility antigens, autoantibodies and lymphopenia or renal disease. Arthritis Rheum, 32:826-836, 1989.
31. Scofield R H, Frank M B, Neas B R, Horowitz R M, Hardgrave K L, Fujisaku A, McArthur R, Harley J B. Cooperative association of T cell receptor $ receptor alleles in the production of anti-Ro in systemic lupus erythematosus. Clin Immunol Immunopathol, 72:335-341, 1994.
32. Reveille J D, MacLeod M J, Whittington K, Arnett F C. Specific amino acid residues in the second hypervariable region of HLA-DQA1 and DQB1 chain genes promote the Ro (SS-A)/La (SS-B) autoantibody responses. J Immunol, 146:3871-3876, 1993.
33. Singh, A K. Lupus in the Fast lane? J R Coll of Physicians (London), 29:475-8. 1995. Review.
34. Wahren, M, Skarstein, K, Blange, I, Pettersson, I, Jonsson, R. MLR/lpr mice produce anti-52,000 MW antibodies: detection, analysis of specificity and site of production. Immunology, 83:9-15, 1994.
35. Kurien B T. Comment on Curcumin attenuates acrylamide-induced cytotoxicity and genotoxicity in HepG2 cells by ROS scavenging. J Agric Food Chem, 57:5644-6, 2009.
36. Kurien B T, Scofield R H. Curcumin/turmeric solubilized in sodium hydroxide inhibits HNE protein modification—an in vitro study. J Ethnopharmacol, 110:368-73, 2007.
37. Kurien B T, Singh A, Matsumoto H, Scofield R H. Improving the solubility and pharmacological efficacy of curcumin by heat treatment. Assay Drug Dev Technol, 5:567-76, 2007.
38. Kurien B T, Jackson K, Scofield R H. Immunoblotting of multiple antigenic peptides. Electrophoresis, 19:1659-61, 1998.
39. Kurien B T, Scofield R H. In vitro modification of solid phase multiple antigenic peptides/autoantigens with 4-hydroxy-2-nonenal (HNE) provide ideal substrates for detection of anti-HNE antibodies and peptide antioxidants. J Immunol Methods, 303:66-75, 2005.
40. Aggarwal B B, Kumar A, Aggarwal M S, Shishodia S. Curcumin derived from turmeric (Curcuma longa): a spice for all seasons. In: Bagchi D, Preuss H G, eds. Phytochemicals in cancer chemoprevention. Boca Raton, Fla.: CRC Press, 349-387, 2004.
41. Bright, J J. Curcumin and autoimmune disease. Adv Exp Med Biology, 595:425-451, 2007. Review.
42. Kurien B T, D'Souza A, Scofield R H. Heat-solubilized curry spice curcumin inhibits antibody-antigen interaction in in vitro studies: A possible therapy to alleviate autoimmune disorders. Mol Nutr Food Res., 54:1202-9. Mol Nutr Food Res. 2010 Feb. 9. [Epub ahead of print]
43. Kurien B T, Dillon S P, Dorri Y, D′ souza A, Scofield R H. Curcumin does not bind or intercalate into DNA and a note on the grey side of curcumin. Int J Cancer, 128:242-5, 2011. Int J. Cancer. 2010 Mar. 2. [Epub ahead of print.]
44. Burgos-Morón E, Calderón-Montaño J M, Salvador J, Robles A, López-Lázaro M. The dark side of curcumin. Int J Cancer, 126:1771-75, 2010.
45. Goodpasture C E, Arrighi F E. Effects of food seasonings on the cell cycle and chromosome morphology of mammalian cells in vitro with special reference to turmeric. Food Cosmet Toxicol, 14:9-14, 1976.
46. Kaur S, Modi N H, Panda D, Roy N. Probing the binding site of curcumin in Escherichia coli and Bacillus subtilis FtsZ—a structural insight to unveil antibacterial activity of curcumin. Eur J Med. Chem., 45:4209-14, 2010.
47. Kurien B T, Scofield R H. Mouse urine collection using clear plastic wrap. Lab Anim, 33:83-86, 1999.
48. Kurien B T, Patel N C, Porter A C, Kurono S, Matsumoto H, Wang H, Scofield R H. Determination of prolidase activity using matrix-assisted laser desorption/ionization time-of-flight mass spectrometry. Anal Biochem., 331:224-29, 2004.
49. Kurien B T, Asfa S, Li C, Dorri Y, Jonsson R, Scofield R H. Induction of oral tolerance in experimental Sjögren's syndrome autoimmunity. Scand J Immunol., 61:418-25, 2005.
50. He J, Zhao J, Li Z. Mucosal administration of alpha-fodrin inhibits experimental Sjögren's syndrome autoimmunity. Arthritis Res Ther, 10:R44, 2008.
51. Kurien B T, Scofield R H. Free radical mediated peroxidative damage in systemic lupus erythematosus. Life Sci, 73:1655-66, 2003.
52. Kurien B T, Scofield R H. Lipid peroxidation in systemic lupus erythematosus. Indian J Exp Biol., 44:349-56, 2006. Review.
53. Kurien B T, Hensley K, Bachmann M, Scofield R H. Oxidatively modified autoantigens in autoimmune diseases. Free Radic Biol Med., 41:549-56, 2006.
54. Gupta A, Vij G, Sharma S., Tirkey N, Rishi P, Chopra K. Curcumin, a polyphenolic antioxidant, attenuates chronic fatigue syndrome in murine water immersion stress model. Immunobiology 214:33-39, 2009.

We claim:

1) A process for solubilizing and stabilizing curcumin in an aqueous solution, comprising:
a) providing a quantity of water;
b) providing over 0.008 mg curcumin per mL water;
c) optionally providing at least 0.01 mL lower alcohol per mL of water;
d) optionally providing at least 0.01 mL polyalkyleneoxide per mL of water;
e) combining the water, curcumin and optional lower alcohol in any order and optionally with mixing; and
f) heating the combined water, curcumin and optional lower alcohol to a temperature in excess of 100%, optionally at a pressure exceeding 1 atmosphere;
wherein steps a), b) and c) may be done in any order, and wherein the process includes at least one of combining the lower alcohol or optional polyalkyleneoxide in step e) and applying a pressure exceeding 1 atmosphere in step f).

2) The process of claim 1 wherein the water provided comprises deionized, distilled, or another quality of water.

3) The process of claim 1 wherein the curcumin is provided in the form of purified curcumin, a mixture of curcuminoids, turmeric, or a portion of rhizome of Curcuma longa.

4) The process of claim 1 wherein the curcumin is provided in a sufficient amount that if it is completely dissolved in a full volume of the provided water, the resulting curcumin concentration exceeds a concentration selected from one of the following: 0.008 mg/mL, 0.015 mg/mL, 0.023 mg/mL, 0.030 mg/mL, 0.038 mg/mL, 0.045 mg/mL 0.052 mg/mL, 0.060 mg/mL, 0.090 mg/mL, 0.12 mg/mL, 0.15 mg/mL, 0.18 mg/mL, 0.24 mg/mL, 0.30 mg/mL, 0.40 mg/mL. 0.50 mg/mL, 0.60 mg/mL, 1.20 mg/mL, 1.80 mg/mL, 2.40 mg/mL and 3.00 mg/mL.

5) The process according to claim 1 wherein the lower alcohol comprises from one to four covalently bonded carbons, is saturated or unsaturated, is linear or cyclic, optionally is branched, optionally is substituted by a halogen, and optionally is a mixture of lower alcohols.

6) The process according to claim 5 wherein the lower alcohol is a saturated alcohol selected from the group consisting of methanol, ethanol, n-propanol, isopropanol, cyclopropanol, n-butanol, sec-butanol, tert-butanol, cyclobutanol, n-hydroxymethyl(cyclopropane), sec-hydroxy(methylcyclopropane) and tert-hydroxy(methylcyclopropane).

7) The process according to claim 5 wherein the alcohol is low boiling.

8) The process according to claim 1 wherein a lower alcohol is provided in a proportion selected from one of the following ranges: up to 75% of the total liquid, 1-50% of the total liquid, 2-25% of the total liquid, 3-15% of the total liquid, 4-10% of the total liquid, or about 5% of the total liquid.

9) The process according to claim 1 wherein ether oxygen atoms of the polyalkyleneoxide (PAO) are separated by from two to six carbons in the backbone of an alkylene group, and wherein the alkylene group is saturated or unsaturated, linear or cyclic, optionally is branched, and optionally is substituted by a halogen.

10) The process according to claim 9 wherein the PAO comprises a mixture of PAO compounds, or is a single PAO compound comprising a mixture of alkylene groups in its structure, or comprises an etherified sugar moiety.

11) The process according to claim 9 wherein the PAO comprises end groups that are hydroxyl, ether, alkyl, or carboxylic ester or comprises internal groups that are ether, alkyl or carboxylic ester.

12) The process according to claim 1 wherein a polyalkyleneoxide is provided in a proportion selected from one of the following ranges: up to 10% of the total liquid, optionally 0.1-8.0% of the total liquid, optionally 0.2-6.0% of the total liquid, optionally 0.5-4.0% of the total liquid, optionally 1-3% of the total liquid, or about 2% of the total liquid.

13) The process according to claim 1 wherein the heating is performed to a maximum temperature selected from within one of the following ranges: 105-300° C., 110-250° C., 115-200° C., 120-150° C., or about 125° C.

14) The process according to claim 1 wherein the pressure exceeding 1 atmosphere is selected from within one of the following ranges: 1-220 atm, 1-100 atm, 1.01-50 atm, 1.01-10 atm, 1.02-5 atm, 1.02-2 atm, or between 1 and 1.2 atm.

15) The process according to claim 1 wherein the heating is performed for a duration selected from within at least one of the following ranges: 10 seconds to 1 week, 1 minute to 72 hours, 5 minutes to 24 hours, 10 minutes to 12 hours, 15 minutes to 6 hours, 20 minutes to 3 hours, 30 minutes to 1 hour, or about 30 minutes.

16) The process according to claim 1 wherein the heating is performed to about 121° C., at a pressure of about 1.02 atm for between 30 and 60 minutes.

17) The process according to claim 1 wherein the heating is carried out in an autoclave, a bomb calorimeter, or a sealed reaction vessel.

18) The process according to claim 1 wherein the heating is conducted in a reaction vessel under pressure from a gas selected from the group consisting of: steam; a noble gas; a relatively inert gas such as carbon dioxide, carbon monoxide, dinitrogen; or a reducing gas such as dihydrogen.

19) The process according to claim 1 wherein the heating is carried out in an unstoppered tube, flask, beaker or other open reaction vessel that is capable of allowing a liquid within it to evaporate and exit the reaction vessel during the heating.

20) The process according to claim 1 wherein the combination is allowed to cool after heating and before further processing.

21) The process according to claim 1 wherein after the water, curcumin and lower alcohol are combined and heated, they are combined with an additional amount of water and or of a lower alcohol to replace fluids lost during heating.

22) The process according to claim 1 wherein after the heating step the solution is filtered to remove undissolved solids, optionally with the assistance of a pump and or a vacuum line.

23) A product comprising solubilized and stabilized curcumin in an aqueous solution made by a process comprising:
a) providing a quantity of water;
b) providing over 0.008 mg curcumin per mL water;
c) optionally providing at least 0.01 mL lower alcohol per mL of water;
d) optionally providing at least 0.01 mL polyalkyleneoxide per mL of water;
e) combining the water, curcumin, optional lower alcohol and optional polyalkyleneoxide in any order and optionally with mixing; and
f) heating the combined water, curcumin and optional lower alcohol to a temperature in excess of 100%, optionally at a pressure exceeding 1 atmosphere;
wherein steps a), b) and c) may be done in any order, and wherein the process includes at least one of combining the lower alcohol or optional polyalkyleneoxide in step e) and applying a pressure exceeding 1 atmosphere in step f).

24) The product of claim 23, wherein the product is further comprised in a food product, a supplement product or a pharmaceutical product.

25) The product of claim 23, wherein the product is further comprised in a beverage, condiment, coloring, glaze, spice, herbal mixture, liquid nutraceutical supplement, gel or capsule.

26) A method of treatment comprising administering to a subject in need thereof an aqueous solution comprising over 0.008 mg curcumin per mL water to treat, prevent or ameliorate a medical or psychological condition.

27) The method of claim 26 wherein amounts of soluble curcumin are administered to a subject to provide a maximum daily selected from the group consisting of up to 2, 4, 6, 8, 10, 12, 14 or 16 g/day of soluble curcumin.

28) The method of claim 26 wherein the subject has a medical condition selected from the group consisting of conditions that require an antioxidative, anti-inflammatory, antiviral, antibacterial and or antifungal effect.

29) The method of claim 26 wherein the subject has a medical condition selected from autoimmune diseases.

30) The method of claim 26 wherein the subject has a medical condition selected from the group consisting of multiple sclerosis, rheumatoid arthritis, psoriasis, inflammatory bowel disease, Sjögren's syndrome, systemic lupus erythematosus, type I diabetes mellitus, neurodegenerative diseases, or a type of cancer.

31) The method of claim 26 wherein the subject has a medical condition selected from the group consisting of Type II diabetes, asthma, allergies, cataracts, atherosclerosis, Alzheimer's disease, Parkinson's disease, myelodysplastic syndromes, cystic fibrosis, myocardial infactions, high cholesterol, stroke, malaria, HIV, HSV-1, and psoriasis.

32) The method of claim 26 wherein the subject has a psychological condition that requires mood elevation.

33) The method of claim 26 wherein the subject has a psychological conditional selected from the group consisting of clinical depression, bereavement, mentally depressing physical pain, dementia, battlefield stress, academic stress, occupational stress, relational stress, homesickness, or other loneliness.

34) The method of claim 26 wherein the subject is a human subject or another mammalian subject.

35) The method of claim 26 wherein the subject is selected from the group consisting of humans, pets, livestock, research animals and display animals.

36) The method of claim 26 wherein the subject is selected from the group consisting of pets such as dogs, cats, rodents, birds, reptiles, amphibians, and fish; livestock such as cattle, horses, sheep, goats, alpaca, chickens, geese, and pigs; research animals such as mice, rats, pigeons, dogs, cats, captive woodland creatures, captive aquatic creatures, captive birds, captive desert creatures, and captive marsh creatures; and display animals such as zoo animals, carnival display animals, and commercial aquarium animals.

37) A process for staining proteins comprising:
a) providing a solution of curcumin that has been solubilized by heating in water, optionally under pressure and optionally in the presence of a lower alcohol or a polyalkyleneoxide;
b) combining the solubilized curcumin with one or more proteins in an assay for an amount of time sufficient to stain the protein; and
c) quantifying an amount of protein as a function of the amount of fluorescence or colorimetric value of the stained protein.

38) The process of claim 37 wherein:
a) the solution of curcumin is obtained from solubilization of turmeric by heating in water, optionally under pressure and optionally in the presence of a lower alcohol or a polyalkyleneoxide;
b) the one or more proteins are comprised in a gel or mini-gel assay format;
c) De-staining of the one or more proteins or the gel or mini-gel is not employed.

39) The process of claim 37 wherein the amount of time for staining is about 30 minutes.

40) The process of claim 37 wherein following the determination step, the curcumin and the one or more proteins with which it is combined is disposed of by any means that is suitable for non-toxic, biodegradable, environmentally friendly compositions.
